(19) United States
(12) Patent Application Publication
(10) Pub. No.: US 2023/0386618 A1
(43) Pub. Date: Nov. 30, 2023

(54) TRAINING AND APPLYING A MACHINE LEARNING MODEL FOR PREDICTING POLYMER EXTRUDATE MELT PROPERTY VALUES

(71) Applicant: Chevron Phillips Chemical Company LP, The Woodlands, TX (US)

(72) Inventors: Chad J. Thomas, Garden Ridge, TX (US); Robert B. Railey, The Woodlands, TX (US); Tom Jarosz, Brooklyn, NY (US); Richa Sethi Chatterjee, Richmond, TX (US); Aaron S. Perez, Houston, TX (US); Sean Beach, Houston, TX (US)

(21) Appl. No.: 18/318,204

(22) Filed: May 16, 2023

Related U.S. Application Data
(60) Provisional application No. 63/365,241, filed on May 24, 2022.

(51) Int. Cl. G16C 60/00

(52) U.S. Cl. CPC G16C 60/00

(57) ABSTRACT

Applying a machine learning model to output predicted melt property values of a polymer extrudate based on an input data set derived from operating parameters of a polymer extruder that produces the polymer extrudate. The machine learning model can also be trained.

### CROSS-REFERENCE TO RELATED APPLICATIONS

This application is a US Non-Provisional patent application that claims benefit of, and priority to, U.S. Provisional Patent Application No. 63/365,241, filed May 24, 2022, and entitled “Training and Applying a Machine Learning Model for Predicting Polymer Extrudate Melt Property Values”, the disclosure of which is hereby incorporated herein by reference as if reproduced in its entirety.

### FIELD OF THE DISCLOSURE

The present disclosure relates to predicting polymer properties with machine learning models, and more particularly, to predicting melt property values of polymer extrudates with machine learning models.

### BACKGROUND

Polymerization reactors implement catalyzed reactions of olefin monomers to produce a polymer product. Examples of polymerization reactors include loop slurry reactors, gas phase reactors (also known as fluidized bed reactors), stirred tank reactors, axial flow reactors, and horizontal gas phase reactors. The polymer product can be withdrawn from the polymerization reactor and subjected to various separations (flashline heating, flashing, degassing, and combinations thereof) to recover the solid polymer called “polymer fluff”.

The polymer fluff can be fed to a polymer extruder that is configured to produce polymer extrudate from the polymer fluff, for example, in the form of polymer pellets. Optional additives can be added to the polymer fluff to impart desired characteristics (e.g., certain mechanical, physical, and melt properties) to the polymer extrudate. The extruder, sometimes referred to as a pelletizer, can convey, heat, melt, and cut the extruder feed, and the molten polymer mixture can be extruded through a pelletizing die under pressure to form the polymer extrudate. The polymer extrudate can then be cooled (e.g., in air or water) at or near the discharge region of the extruder. The polymer extrudate may then be transported to a product load-out area for further use such as storing, blending with other pellets, and/or loading into railcars, trucks, bags, supersacks, or other containers for distribution to customer(s).

Polymer manufacturers desire to monitor the properties of the polymer extrudate that is produced, for example, to verify that the product sold to a customer is within requested specifications.

One technique for monitoring polymer extrudate properties is asynchronous testing of samples of the polymer extrudate for melt property values. A rheometer can be used to test samples of the polymer extrudate after the polymer extrudate is formed. However, extrudate production and extrudate testing are asynchronous (i.e., occur at different points in time), and as such, production of polymer extrudate can be significantly disrupted when off-spec melt property values are obtained. For example, if the polymer extrudate has an off-spec melt property value determined at a point in time that is after production of the extrudate, then the manufacturer must determine how much of the extrudate is off-spec, determine how long the extrudate was produced off-spec, and separate the off-spec product from on-spec product. In some cases, a manufacturer may even have to perform another polymerization run to produce product that meets specifications.

Another technique for monitoring polymer extrudate properties utilizes in-line melt property measurement devices to provide real-time melt property values of the polymer extrudate. This technique avoids the issues involved with asynchronous testing of extrudate; however, such devices have previously provided unreliable data and require extruder downtime to make repairs.

There is an ongoing need for reliably determining melt property values of polymer extrudate.

### SUMMARY

Disclosed herein is a method and computer for applying, while a polymer extruder produces a first polymer extrudate, a machine learning model to an input data set to output a predicted melt property value for the first polymer extrudate, wherein the input data set includes a raw value data point for each of a plurality of operating parameters of the polymer extruder at a first point in time. In some aspects, the input data set can also include a delta value for each of the plurality of operating parameters, wherein the delta value is a difference between the raw value data point at the first point in time and a previous raw value data point for each of the plurality of operating parameters of the polymer extruder at a second point in time. In some aspects, the input data set can include a measured melt property value for a sample of a second polymer extrudate obtained before the first point in time.

Disclosed herein is a method and computer for training a machine learning model to output predicted melt property values of a polymer extrudate using a training data set. In some aspects, the training data set can include i) a measured melt property value for the sample; ii) a first plurality of operating data points for a plurality of operating parameters of the polymer extruder corresponding to when the sample was collected; and iii) a first plurality of delta values corresponding to a difference between the first plurality of operating data points and a second plurality of operating data points of the polymer extruder, wherein the second plurality of operating data points corresponds to a previous sample that was collected from the polymer extruder before the sample was collected. In other aspects, the training data set can include i) a first plurality of average value data points for a plurality of operating parameters of a polymer extruder, and ii) a plurality of measured melt property values corresponding to a plurality of samples of a polymer extrudate obtained from the polymer extruder.

Other technical features may be readily apparent to one skilled in the art from the following figures, descriptions and claims.

### BRIEF DESCRIPTION OF THE DRAWINGS

For a more complete understanding of this disclosure, reference is now made to the following description, taken in conjunction with the accompanying drawings, in which:

FIG. 1 illustrates a block diagram of a polymer extrusion system according to the disclosure.

FIG. 2 illustrates a side elevational view of an embodiment of the polymer extruder of FIG. 1.

FIG. 3 illustrates a flow diagram of a method for training and applying a machine learning model to output predicted melt property values for a polymer extruder.

FIG. 4 illustrates a flow diagram of a method for obtaining a training data set used to train a machine learning model according to the disclosure.

FIG. 5 is a schematic diagram illustrating how average values are calculated when samples are made by combining portions of polymer extrudate that are collected over a sample collection frequency.

FIG. 6 is a graph of the ratio of HLMI versus time showing a solid line for actual values and a dashed line for values predicted with a machine learning model, where delta values were not used in the input data set.

FIG. 7 is a graph of the ratio of HLMI versus time showing a solid line for actual values and a dashed line for values predicted with a machine learning model, where delta values were used in the input data set.

FIG. 8 is a graph of the ratio of MI versus time showing a solid line for actual values and a dashed line for values predicted with a machine learning model, where the delta values were based on 2 hours between the data points for purposes of calculating the delta values.

FIG. 9 is a graph of the ratio of MI versus time showing a solid line for actual values and a dashed line for values predicted with a machine learning model, where the delta values were based on 4 hours between the data points for purposes of calculating the delta values.

### DETAILED DESCRIPTION

It should be understood at the outset that although an illustrative implementation of one or more embodiments are provided below, the disclosed computer system, computer, and/or method may be implemented using any number of techniques, whether currently known or in existence. The disclosure should in no way be limited to the illustrative implementations, drawings, and techniques illustrated below, including the exemplary designs and implementations illustrated and described herein, but may be modified within the scope of the appended claims along with their full scope of equivalents.

“Melt property value” as used herein refers to a value for a rheological property of a polymer. A rheological property can be measured using a rheometer. The rheometer can determine the melt viscosity of the polymer at any shear rate, and the viscosity data can be correlated to determine a melt index value (e.g., melt index (MI₂) value, melt index (MI₅) value, melt flow (MF) value, high load melt index (HLMI) value, or other value) of the polymer. Measurements can be obtained, for example, using proprietary testing procedures, non-standard testing procedures, or standardized testing procedures such as those found in ASTM D1238 and ISO 1133. The viscosity data can be determined at any shear rate, including but not limited to 1/0.01, 1/0.1, 1/0.5, 1/100, 1/500 reciprocal seconds. Viscosity at zero shear rate can also be determined.

“Polymer extrudate” refers to a solid product that is formed by melting polymer fluff in an extruder, adding any additives to the polymer fluff or melted polymer flowing in the extruder, and cooling the melt into shaped objects to form the polymer extrudate. The shaped objects can be referred to as pellets.

“Real-time” as used herein can refer to a speed of data transfer and data processing when transfer and processing in online. “Real-time” can include “near real-time” conditions where lags occur, and the exact time of data transfer or processing is not instantaneous because of computing and data transfer limitations that can occur with computing and networking equipment. For example, real-time signals sent by sensors in real-time and received by an extruder data computer system in real-time as described herein includes any lag in time associated with converting sensed conditions to a signal that can be transferred to a computer component, and any lag in signal transfer between computer components. In another example, real-time transfer of real-time extruder data can include, without being limited to, any lags associated with data passing through networking equipment, lags associated with packaging data into packets for transfer, lags associated with any encryption and decryption, etc. In another example, reference to a database as a real-time extrusion database can include any lag for storing data and passing data through the computer components of the database to another computer such as the melt property prediction computer disclosed herein.

“Supervised” as used herein with reference to training a machine learning model refers to labels being assigned to data in the training data set and the training output data set so as to measure the accuracy of the machine learning model.

Disclosed are methods and computers for training and applying a machine learning model to output polymer extrudate melt property value predictions. Also disclosed herein are methods and computers that can provide real-time melt property prediction of polymer extrudate by applying the trained machine learning model to an input data set derived from operating parameter values of the polymer extruder. Training of the machine learning model and applying the machine learning model do not utilize or rely on direct in-line measurements of melt property values of polymer extrudate, and yet, provide accurate real-time predicted melt property values.

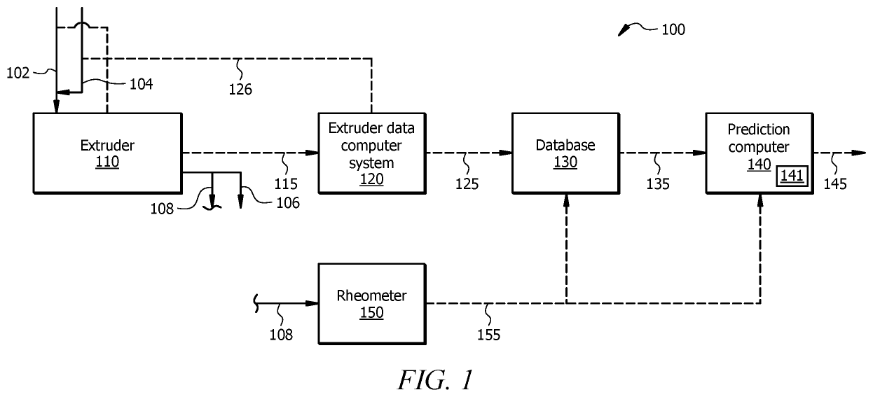

FIG. 1 illustrates a block diagram of a polymer extrusion system 100 according to the disclosure. The polymer extrusion system 100 of FIG. 1 provides polymer extrudate, provides a training data set to train a machine learning model, and provides predicted melt property values for the polymer extrudate using the trained machine learning model. The polymer extrusion system 100 can include one or more of a polymer extruder 110, an extruder data computer system 120, a database 130, a melt property prediction computer 140, and a rheometer 150. The polymer extruder 110 can be networked with the extruder data computer system 120, the extruder data computer system 120 can be networked with the database 130, the database 130 can additionally be networked with the melt property prediction computer 140. Embodiments contemplate that the rheometer 150 can be networked with the database 130, the melt property prediction computer 140, or both.

Each of the components 120, 130, and 140 shown in FIG. 1 can be embodied with computer equipment such as one or more processors, memory, networking cards or interfaces, and other equipment for receiving, processing, and sending data according to the functionality described herein.

The networking between any two of components 110, 120, 130, 140, and 150 of the polymer extrusion system 100 can be embodied as any wired internet connection, wireless internet connection, local area network (LAN), wired intranet connection, wireless intranet connection, or combinations thereof. Wireless internet connections can include a Global System for Mobile Communications (GSM), Code-division multiple access (CDMA), General Packet Radio Service (GPRS), Evolution-Data Optimized (EV-DO), Enhanced Data Rates for GSM Evolution (EDGE), Universal Mobile Telecommunications System (UMTS), or combinations thereof.

The polymer extruder 110 can be embodied as any polymer extruder known in the art with the aid of this disclosure. Polymer fluff can be continuously fed to the polymer extruder 110 via a feed line 102, along with one or more optional additives that are added to the feed line 102 via additive line 104.

The polymer extruder 110 can be configured to receive the polymer fluff and any additives, produce a molten blend of the fluff and additive(s), and produce the polymer extrudate. The polymer extrudate in this discussion can be embodied as pellets. Polymer extrudate is illustrated as flowing out of the polymer extruder 110 in transfer line 106; however, in practice, the polymer extrudate may fall out of the polymer extruder 110 or otherwise be pushed out of the polymer extruder 110 by upstream flow of molten polymer and polymer extrudate, into a transfer line 106. Transfer line 106 can be embodied as a conveyor, chute, pipe, or combinations thereof, for example. The polymer extrudate can be transferred directly into a container, such as a rail car, or can be subjected to polymer extrudate processing (e.g., drying) prior to being ultimately transferred to a container.

In embodiments, the polymer extruder 110 is configured to have a production rate of polymer extrudate greater than 5,000, 10,000, 20,000, 50,000, 100,000, 125,000, 200,000, or 300,000 lb/hr.

A portion of the polymer extrudate can be continuously or periodically (e.g., every 1, 2, 3, 4, 5, 6, 7, 8, 9, or 10 minutes) recovered from the polymer extrudate that flows from the polymer extruder 110. The recovered portion is shown in sampling line 108 that is connected to transfer line 106; however, it is contemplated that the portion of polymer extrudate can be recovered by any other mechanism, such as through a screen that selectively allows only a portion of the polymer extrudate to be separated from the main flow of polymer extrudate product out of the polymer extruder 110, or such as a line connected to transfer line 106 that has a solenoid valve configured to periodically actuate to allow flow of polymer extrudate therethrough.

Nonlimiting examples of additives include surface modifiers, slip agents (such as oleamide, erucamide, stearamide, behenannfle, oleyl paimitamide, stearyl erucamide, ethylene bis-oleamide, N,N′-Ethylene Bis(Stearamide) (EBS), including most grades of their respective refinement), antiblocks/anti-block agents (also called “antitack” agents) such as diatomaceous earth, tackifiers, dispersing agents, antioxidants, nucleating agents, pigments, dyes and colorants, including TiO2, processing aids such as elastomers, waxes, oils, fluoroelastomers, antistats/anti-static agents, scavengers, odor enhancers, degradation agents, ultraviolet stabilizers, heat stabilizers, viscosity enhancers, plasticizers, delustrants, flame retardants such as antimony oxide, fillers and extenders such as alumina, silica, clays, and calcium carbonate, surfactants, lubricants such as talc, glass fibers, blowing agents, and combinations thereof.

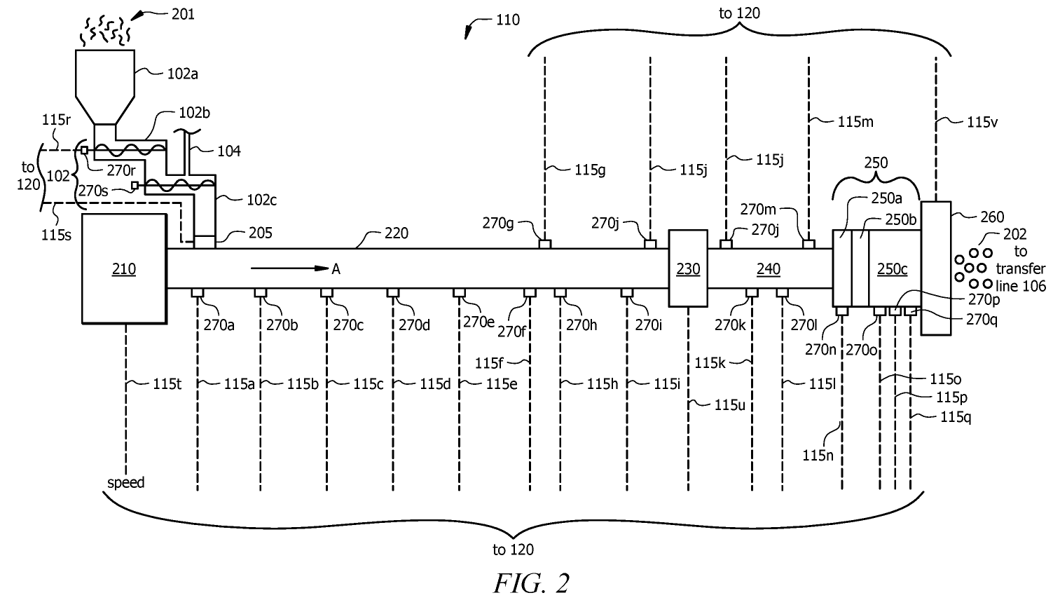

The polymer extruder 110 can generally include sensors that are configured to send signals 115 indicating extruder operating parameter values associated with extruding polymer fluff into a polymer extrudate to the extruder data computer system 120 on a continuous basis. The sensors are generally coupled to the extruder and networked with the extruder data computer system 120. The sensors are configured to send the signals 115 to the extruder data computer system 120, in real-time. Signals 115 for the operating parameters that can be sensed and collected by the extruder data computer system 120 can include signals for master feeder counts, a polymer fluff feed rate to the polymer extruder 110, a speed of a drive motor of the polymer extruder 110, one or more temperatures in one or more zones of the screw portion of the polymer extruder 110, a polymer melt temperature in one or more zones of the screw portion of the polymer extruder 110, one or more pressures in one or more zones of the screw portion of the polymer extruder 110, one or more temperatures in one or more zones of the melt flow portion of the polymer extruder 110, a temperature at a die plate of the polymer extruder 110, a pressure at the die plate of the polymer extruder 110, a polymer melt temperature at the die plate of the polymer extruder 110, a speed of the pelletizer of the polymer extruder 110, a differential pressure across the screenpack of the polymer extruder 110, at least one bearing temperature of a gear pump of the polymer extruder 110, a temperature of the oil of the gear pump of the polymer extruder 110, a suction pressure of the gear pump, a discharge pressure of the gear pump, an oil temperature of the gear pump, a speed of the gear pump, an amperage of the gear pump (e.g., indicative of speed), or combinations thereof. Additional description for the polymer extruder 110 is provided for the embodiment of the polymer extruder 110 that is illustrated in FIG. 2.

The extruder data computer system 120 generally includes computer(s) and networking infrastructure that are configured to monitor, control, record, or combinations thereof, extruder operation parameters associated with extruding polymer fluff into a polymer extrudate on a continuous basis. The computer of the extruder data computer system 120 can generally include one or more processors and one or more memory having instructions stored thereon that cause the one or more processors to receive and detect the real-time signals from the sensors coupled to the polymer extruder 110. The computer of the extruder data computer system 120 is configured to convert the signals to data values associated with particular extruder operating parameters and apply a time stamp to each data value for each parameter. The computer of the extruder data computer system 120 is also configured to send the data values of the operating parameters that are time stamped to the database 130 as a stream 125 of data that is referred to herein as time-series real-time extruder data. The format of the data values in the time-series real-time extruder data can be any format known in the art with the aid of this disclosure, such as XML Format, Hierarchical Data Format (HDF), Excel Format, Java Script Object Notation (JSON), Statistical Package for the Social Sciences (SPSS), Comma-Separated Values (CSV), Apache Parquet, or combinations thereof.

In some optional aspects, the computer of the extruder data computer system 120 can be configured to also send the stream 125 of the time-series real-time data to the melt property prediction computer 140 or to allow the melt property prediction computer 140 to retrieve time-series real-time extrusion data from one or more datastores of the database 130.

In additional aspects, the extruder data computer system 120 can additionally be coupled to components of the polymer extruder 110 via control line 126 to control operation of the polymer extruder 110, such as polymer fluff feed rate in line 102, additive feed rate in line 104, or operating parameters of the polymer extruder 110 such as drive motor speed, gear pump speed, pellet production rate, screw portion temperature, differential pressure across the die plate, die plate temperature, or combinations thereof.

The database 130 is a real-time extrusion database configured to store the stream 125 of time-series real-time extrusion data that is received from the extruder data computer system 120 in any format known in the art with the aid of this disclosure. The database 130 can generally include one or more processors, one or more datastores, and one or more memory having instructions stored thereon that cause the one or more processors to store the time-series real-time extrusion data in the one or more datastores. The database 130 can be located entirely in the cloud, partially in the cloud (e.g., having portions on the edge and/or in locally stored datastore), or entirely local.

For melt property value prediction, the database 130 can be configured to send a stream 135 of the time-series real-time extrusion data to the melt property prediction computer 140 or to allow the melt property prediction computer 140 to retrieve time-series real-time extrusion data from the one or more datastores of the database 130. In embodiments, the database 130 simultaneously stores the stream 125 of the time-series real-time extrusion data in the one or more datastores and sends the stream 135 to the melt property prediction computer 140.

For training of a machine learning model, the database 130 can be configured to send time-series real-time extrusion data to the melt property prediction computer 140 or to allow the melt property prediction computer 140 to retrieve time-series real-time extrusion data from the one or more datastores of the database 130.

The melt property prediction computer 140 is configured to train one or more machine learning models 141 stored on the melt property prediction computer 140 and to apply the trained machine learning model(s) 141 to the stream 135 of time-series real-time extrusion data. The machine learning model(s) 141 can include a decision tree-based model, a K nearest neighbor (KNN) model, a neural network model, a stochastic gradient descent linear model, or combinations thereof. In aspects, the machine learning model(s) is supervised. In embodiments, the decision tree-based model is an ensemble machine learning model. An example of a decision-tree based ensemble machine learning model is a gradient-boosting decision tree model, such as Extreme Gradient Boosting model (XGBoost).

The melt property prediction computer 140 can be configured to receive or retrieve the stream 135 of time-series real-time extrusion data from the database 130 (or receive or retrieve the stream 125 of time-series real-time extrusion data from the extruder data computer system 120) and to output a melt property value prediction(s) (e.g., to a display of the melt property prediction computer 140). The melt property prediction computer 140 can generally include one or more processors, one or more datastores, and one or more memory having instructions stored thereon that cause the one or more processors to process the stream 125 or 135 of time-series real-time extrusion data such that the time-series real-time extrusion data is converted to a stream 145 containing one or more of melt property value predictions.

The rheometer 150 can be embodied as any commercially available rheometer known in the art for determining a melt property value of a polymer. It should be understood that, in practice, portions of polymer extrudate may be collected in a container, and a collection of portions of polymer extrudate that are obtained over an interval of time that is the sample collection frequency may be blended and physically moved to a location of the rheometer 150 for testing of a measured melt property value.

The rheometer 150 can include one or more processors, one or more datastores, one or more networking cards, and one or more memory having instructions stored thereon that cause the one or more processors to send a measured melt property value 155 to the database 130, to the melt property prediction computer 140, or both. The measured melt property value 155 corresponds to the sample (containing one or more portions) of polymer extrudate obtained from the polymer extruder 110. Alternatively, the rheometer 150 can include a display that displays the measured melt property value 155, and a technician or other personnel can enter the measured melt property value 155 into the database 130, the melt property prediction computer 140, or both.

FIG. 2 illustrates a side elevational view of an embodiment of the polymer extruder 110 of FIG. 1. The polymer extruder 110 is configured to cover polymer fluff 201 into polymer extrudate, which in FIG. 2 is embodied as pellets 202. In embodiments, the polymer extrudate, e.g., pellets 202, is a homopolymer or copolymer of one or more olefin monomers. The components of the extruder 110 are not drawn to scale and are shown in certain proportions for purposes of illustration and description in this disclosure.

The extruder 110 can have an inlet 205, a drive motor 210, a screw portion 220, a gear pump 230, a molten flow portion 240, a die plate assembly 250, and a pelletizer 260, connected as shown in FIG. 2. Alternative embodiments contemplate that the extruder 110 can be embodied without a gear pump 230. In such embodiments, the sensors and signals disclosed herein that are associated with the gear pump 230 are not utilized in the training data set since the extruder in such alternative embodiments does not include a gear pump.

The feed line 102 is connected to the inlet 205 of the polymer extruder 110. In FIG. 2, the feed line 102 has a hopper 102a, a master fluff feeder line 102b connected to an outlet of the hopper 102a, and a second fluff feeder line 102c connected to an outlet of the master fluff feeder line 102b and to the inlet 205 of the polymer extruder 110. The master fluff feeder line 102b has a rotating auger that moves polymer fluff from the hopper 102a to the second fluff feeder line 102c. The second fluff feeder line 102c also has at least one rotating auger to move polymer fluff and any additives added via line 104 to the inlet 205 of the polymer extruder 110, and into the screw portion 220 of the polymer extruder 110.

The drive motor 210 is connected to at least one extruding screw that rotates inside the screw portion 220 so as to move the polymer fluff or blend of polymer fluff in the direction of arrow A as heat is applied (e.g., via external heating source such as electric heater or steam heating jacket around the screw portion 220) to the outer surface of the screw portion 220.

In embodiments of extruder 110 having a gear pump 230 such as that illustrated in FIG. 2, the gear pump 230 receives molten polymer and pumps molten polymer into the molten flow portion 240 of the extruder, and then into the die plate assembly 250. Other embodiments of polymer extruder 110 that do not utilize a gear pump 230 are contemplated. In those embodiments, the molten polymer can flow from the screw portion 220 to the die plate assembly 250.

The die plate assembly 250 has (in the direction of polymer flow) a screenpack 250a, followed by a die plate approach 250b, and a die plate 250c. In the die plate assembly 250, the molten polymer cools into the shape provided by the die plate 250c. The cooled polymer then enters the pelletizer 260 that is configured to cut the cooled polymer in the polymer extrudate, which in FIG. 2, is pellets 202.

The polymer extruder 110 includes various sensors placed in various components to measure various operating parameters of the polymer extruder 110. Some components, such as the drive motor 210, the gear pump 230, and the pelletizer 260, may have sensors, or devices that produce signals which can be interpreted for measurements, included within the components to measure operating parameters.

The operating parameters of the polymer extruder 110 (which can also be referred to generally as variables, attributes, or features) that be included in the training data set disclosed herein include i) counts measured in the master feed line 102b, ii) the extruder fluff feed rate measured by the flow meter in inlet 205, iii) a speed of the drive motor 210, iv) one or more temperatures in one or more zones of the screw portion 220, v) one or more temperatures of the polymer (e.g., polymer melt or molten polymer) in one or more zones of the screw portion 220), vi) a pressure in one or more zones of the screw portion 220, vii) one or more temperatures in one or more zones of the melt flow portion 240, viii) a temperature for at least one bearing (or each bearing) of the gear pump 230, ix) a temperature of the oil of the gear pump 230 (e.g., oil temperature of a finishing gear), x) an amperage of the gear pump 230, xi) a speed of the gear pump 230, xii) a suction pressure of the gear pump 230, xiii) a discharge pressure of the gear pump 230, xiv) a screenpack 250a differential pressure, xv) a temperature of the die plate 250c, xvi) a temperature of the polymer in the die plate 250c, xvii) a pressure in the die plate 250c, xviii) a speed of the pelletizer 260, xix) a ratio of power to amperage of the gear pump 230, or xx) combinations thereof.

Sensors 270a, 270b, 270c, 270d, 270e, 270f, and 270i are thermocouples placed on the screw portion 220 of the polymer extruder 110. That is, sensors 270a, 270b, 270c, 270d, 270e, 270f, and 270i can be placed on an outer surface of the screw portion 220 (or on the heat source that is on the outer surface of the screw portion 220) to measure the temperature. Sensor 270a is a thermocouple in a first zone of the screw portion 220 that produces signal 115a. Sensor 270b is a thermocouple in a second zone of the screw portion 220 that produces signal 115b. Sensor 270c is a thermocouple in a third zone of the screw portion 220 that produces signal 115c. Sensor 270d is a thermocouple in a fourth zone of the screw portion 220 that produces signal 115d. Sensor 270e is a thermocouple in a fifth zone of the screw portion 220 that produces signal 115e. Sensor 270f is a thermocouple in a sixth zone of the screw portion 220 that produces signal 115f. Sensor 270i is a thermocouple in a seventh zone of the screw portion 220 that produces signal 115i.

The first through seventh zones can be located in the screw portion 220. The first zone is upstream of the second zone, the second zone is upstream of the third zone, the third zone is upstream of the fourth zone, the fourth zone is upstream of the fifth zone, the fifth zone is upstream of the sixth zone, and the sixth zone is upstream of the seventh zone. The sixth zone can be a zone of the screw portion 220 that is proximate a throttle valve that is placed in the screw portion 220. The seventh zone is the final zone of the screw portion 220 that is fluidly connected to the inlet of the gear pump 230.

Alternative embodiments contemplate that more or fewer zones may be present in a screw portion 220 of the polymer extruder 110. Alternative embodiments also contemplate that the final zone of screw portion 220 can be connected directly to an inlet of the die plate assembly 250.

Sensor 270g is a thermocouple placed in the sixth zone of the screw portion 220 so as to measure a temperature of the polymer (e.g., molten polymer or polymer melt). Sensor 270g produces signal 115g that is indicative of the temperature.

Sensor 270h is a pressure transducer placed in the sixth zone of the screw portion 220 so as to measure a pressure of the polymer (e.g., molten polymer or polymer melt). Sensor 270h produces signal 115h that is indicative of the pressure.

Sensors 270j are pressure transducers configured to measure the suction pressure and discharge pressure of the gear pump 230, and produce signals 115j indicative of the suction and discharge pressures.

Sensors 270k, 2701, and 270m are thermocouples placed on the molten flow portion 240 of the polymer extruder 110. That is, sensors 270k, 2701, and 270m can be placed on an outer surface of the molten flow portion 240 (or on the heat source that is on the outer surface of the molten flow portion 240) to measure the temperature. Sensor 270k is a thermocouple in a first zone of the molten flow portion 240 (e.g., an eighth zone of the polymer extruder 110) that produces signal 115k. Sensor 270l is a thermocouple in a second zone of the molten flow portion 240 (e.g., a ninth zone of the polymer extruder 110) that produces signal 115l. Sensor 270m is a thermocouple in a third zone of the molten flow portion 240 (e.g., a tenth zone of the polymer extruder 110) that produces signal 115m.

The first zone of the molten flow portion 240 can be fluidly connected to an outlet of the gear pump 230. The second zone of the molten flow portion 240 can be downstream of the first zone and upstream of the third zone of the molten flow portion 240. The third zone of the molten flow portion 240 can be downstream of the first and second zones and fluidly connected to an inlet of the die plate assembly 250.

Sensor 270n is a differential pressure sensor that measures a differential pressure across the screenpack 250a of the die plate assembly 250. The differential pressure is indicated in signal 115n.

Sensor 270o is a thermocouple that measures a temperature of the die plate 250c, and signal 115o is indicative of the temperature. Sensor 270p is a thermocouple that measures a temperature of the polymer in the die plate 250c, and signal 115p is indicative of the temperature. Sensor 270q is a pressure transducer that measures a pressure of the polymer in the die plate 250c, and signal 115q is indicative of the pressure.

Alternative embodiments of the polymer extruder 110 contemplate that there is no gear pump; thus, alternative embodiments may have the screw portion 220 directly connected to the inlet of the die plate assembly 250. In such alternative embodiments, no molten flow portion 240 is present, and thus, the sensors 270j, 270k, 270, and 270m (along with accompanying signals 115j to 115m) would also not be present.

Sensor 270r can be a speed sensor to measure a speed of the auger in the master feeder line 102b. The sensor 270r can produce a signal 115r indicative of the speed of the auger.

Sensor 270s can be a speed sensor to measure a speed of the auger(s) in the master feeder line 102c. The sensor 270s can produce a signal 115s indicative of the speed of the auger.

The inlet 205 can be embodied as or include a flow meter that can produce a signal 115s indicative of the extruder fluff feed rate.

The drive motor 210 can include various sensors or other devices for producing signals, collectively shown in signal 115t, that are indicative of the speed of the drive motor 210. For example, signal 115t can be indicative of a high speed and a low speed, or be embodied as two signals where one signal is indicative of a high speed and another signal is indicative of a low speed.

The gear pump 230 can include various sensors or other devices for producing signals, collectively shown in signal 115u, that are indicative of i) a gear pump bearing temperature (e.g., a temperature signal for each bearing of the gear pump, e.g., a sensor and signal for each bearing), ii) an oil temperature of the gear pump 230, iii) a speed of the gear pump 230, iv) an amperage of the gear pump 230, or v) combinations thereof.

The pelletizer 260 can include various sensors or other devices for producing signals, collectively shown in signal 115v, that are indicative of the pelletizer speed.

The signals 115a to 115v illustrated in FIG. 2 (collectively shown in FIG. 1 as signals 115) are received by the extruder data computer system 120 for processing, analysis, control, and use as described herein.

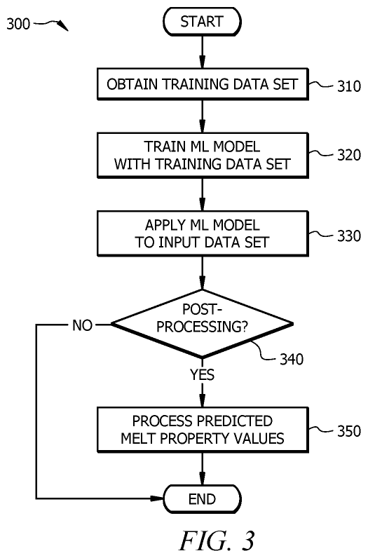

FIG. 3 illustrates a flow diagram of a method 300 for training and applying a machine learning model to output predicted melt property values for a polymer extruder. It is contemplated that the melt property prediction computer 140 performs the method 300, unless noted herein. Steps of the method 300 are described with reference to components of the polymer extrusion system 100 in FIG. 1.

At block 310, the method 300 includes obtaining a training data set. The training data set generally includes data values regarding polymer extrudate made by the polymer extruder 110 and operating parameters of the polymer extruder 110. Thus, obtaining the training data set includes operating the polymer extruder 110 to produce a polymer extrudate, where the operating parameters of the polymer extruder 110 are recorded and stored, and samples of the polymer extrudate are measured for melt property values. Any or all of features and feature values in the training data set can be historical and can be entered, retrieved from a datastore, or otherwise obtained to build the training data set by an administrator or user of the prediction computer 140 and associated with the respective samples and intervals of time. The time period over which the operating parameters are collected and recorded is the same time period over which the polymer extrudate(s) was/were made and samples collected.

In some aspects, the training data set can include for each sample of a polymer extrudate obtained from the polymer extruder 110: i) a measured melt property value corresponding to the sample, and ii) an average value data point or raw value data point for each of the following operating parameters of the polymer extruder 110:
i) counts measured in the master feed line 102b,
ii) the extruder fluff feed rate measured by the flow meter in inlet 205,
iii) a speed of the drive motor 210,
iv) one or more temperatures in one or more zones of the screw portion 220,
v) one or more temperatures of the polymer (e.g., polymer melt or molten polymer) in one or more zones of the screw portion 220),
vi) a pressure in one or more zones of the screw portion 220,
vii) one or more temperatures in one or more zones of the melt flow portion 240,
viii) a temperature for at least one bearing (or each bearing) of the gear pump 230,
ix) a temperature of the oil of the gear pump 230 (e.g., oil temperature of a finishing gear),
x) an amperage of the gear pump 230,
xi) a speed of the gear pump 230,
xii) suction pressure of the gear pump 230,
xiii) a discharge pressure of the gear pump 230,
xiv) a screenpack 250a differential pressure,
xv) a temperature of the die plate 250c,
xvi) a pressure in the die plate 250c,
xvii) a speed of the pelletizer 260,
xviii) a temperature of the polymer in the die plate 250c, or
xix) combinations thereof.
   The average value for each of the above operating parameters is utilized when the sample is collected over an interval of time (as opposed to a point in time); whereas, the raw value for each of the above operating parameters is utilized when the sample is collected at a single point in time.

In these aspects, the training data set can optionally include, for each sample, the type of the polymer (e.g., homopolymer, copolymer, polyethylene, ethylene-butene-copolymer, etc.) associated with the measure melt property value and associated with the average value data points or raw value data points, and an identifier for the polymer extruder 110 that produced the sample. In these aspects, it is contemplated that the training data set can include multiple sub sets of measured melt property values and average value data points corresponding to samples obtained from different extruders, samples obtained for different types of polymer, and samples obtained for different types of polymers from the same extruder, and samples obtained for different types of polymers from different extruders.

In additional or alternative aspects, the training data set can include, for each sample of a polymer extrudate obtained from the polymer extruder 110: the type of the polymer (e.g., homopolymer, copolymer, polyethylene, ethylene-butene-copolymer, etc.), the measured melt property value corresponding to the sample, a value for the interval of time that elapsed since a previous sample was collected before the sample began being collected, an identifier for the polymer extruder 110, an average value or raw value for each of the following operating parameters:
i) counts measured in the master feed line 102b,
ii) the extruder fluff feed rate measured by the flow meter in inlet 205,
iii) a speed of the drive motor 210,
iv) one or more temperatures in one or more zones of the screw portion 220,
v) one or more temperatures of the polymer (e.g., polymer melt or molten polymer) in one or more zones of the screw portion 220),
vi) a pressure in one or more zones of the screw portion 220,
vii) one or more temperatures in one or more zones of the melt flow portion 240,
viii) a temperature for at least one bearing (or each bearing) of the gear pump 230,
ix) a temperature of the oil of the gear pump 230 (e.g., oil temperature of a finishing gear),
x) an amperage of the gear pump 230,
xi) a speed of the gear pump 230,
xii) suction pressure of the gear pump 230,
xiii) a discharge pressure of the gear pump 230,
xiv) a screenpack 250a differential pressure,
xv) a temperature of the die plate 250c,
xvi) a pressure in the die plate 250c,
xvii) a speed of the pelletizer 260,
xviii) a temperature of the polymer in the die plate 250c,
xix) polymerization reactor polymer production rate,
xx) a ratio of power to amperage of the gear pump 230, or
xxi) combinations thereof,
   and a change in value or “delta value” for the each of the above operating parameters over the interval of time. In some aspects, examples of the delta values can include:
i) a delta value for the pressure in the die plate 250c,
ii) a delta value for the temperature of the polymer in the die plate 250c, iii) a delta value for the speed of the drive motor 210,
iv) a delta value for the extruder fluff feed rate,
v) a delta value for the temperature of the oil of the gear pump 230 (e.g., oil temperature of a finishing gear),
vi) a delta value for the temperature for at least one bearing (or each bearing) of the gear pump 230,
vii) a delta value for the amperage of the gear pump 230,
viii) a delta value for the discharge pressure of the gear pump 230,
ix) a delta value for the suction pressure of the gear pump 230,
x) a delta value for the speed of the gear pump 230,
xi) a delta value for the ratio of power to amperage of the gear pump 230,
xii) a delta value for the counts measured in the master feed line 102b,
xiii) a delta value for the speed of the pelletizer 260,
xiv) a delta value for the polymerization reactor polymer production rate,
xv) a delta value for the screenpack 250a differential pressure
xvi) a delta value for one or more temperatures in one or more zones of the screw portion 220,
xvii) a delta value for one or more temperatures of the polymer (e.g., polymer melt or molten polymer) in one or more zones of the screw portion 220),
xviii) a delta value for the pressure in one or more zones of the screw portion 220, or
xix) combinations thereof.

The average value for each of the above operating parameters is utilized when the sample is collected over the enter interval of time; whereas, the raw value for each of the above operating parameters is utilized when the sample is collected at a single point in time, and the interval of time is defined as the amount of time elapsed between the single point and time at which the sample was collected and a previous point in time (which can be an endpoint of a previous interval of time) at which a previous sample was collected or finished being collected.

In these additional or alternative aspects for the training data set, it is contemplated that the training data set can include multiple sub sets. First, the training data set includes a combination of the above feature values for each sample. Samples can include samples from different types of polymers, samples obtained from different extruders, samples obtained for different types of polymers from the same extruder, and samples obtained for different types of polymers from different extruders. Second, the training data set can include sub sets for different time intervals before or after collection of a sample. For example, four samples can be collected in series, a first sample, then a second sample, then a third sample, and then a fourth sample. Each of the first, second, third, and fourth sample includes the associated parameters values associated with the point in time in which the respective sample was collected or the interval of time over which the sample was collected. A first subset of training data can be the parameter values (including the delta values) for the first sample relative to the second sample, a second subset of training data can be the parameters values for the second sample relative to the third sample, a third subset of training data can be the parameters values for the third sample relative to the fourth sample, a fourth subset of training data can be the parameters values for the first sample relative to the third sample, a fifth subset of training data can be the parameters values for the first sample relative to the fourth sample, and a sixth subset of training data can be the parameters values for the second sample relative to the fourth sample. In an example where the interval of time between the first and second samples is 2 hours, the second the third samples is 1 hour, and third and fourth samples is 3 hours; the interval of time for the first subset of training data is 2 hours, the interval of time for the second subset of training data is 1 hour, the interval of time for the third subset of training data is 3 hours, the interval of time for the fourth subset of training data is 3 hours, the interval of time for the fifth subset of training data is 6 hours, and the interval of time for the sixth subset of training data is 4 hours. In these additional and alternative aspects of the training data set, the intervals of time for sample collection do not have to be the same and can be different.

The average value data points, when present in the training data set, correspond to time-series data for the operating parameters of the polymer extruder 110 that is generated while the polymer extruder 110 forms the polymer extrudate. In further aspects, each of the average value data points is an average value for a time-series data set for one of the operating parameters that is collected over the interval of time since the previous sample was collected. In aspects, the average value data points can include average values determined at a calculation frequency that is equal to the interval of time. In further aspects, the calculation frequency is equal to a sample collection frequency for each of the samples of the polymer extrudate.

At block 320, the method 300 includes training a machine learning (ML) model with the training data set. The training data set obtained in block 310 is used as an input data set to train the ML model(s) that are on the melt property prediction computer 140. The training data set is input to the ML model(s), the ML model(s) are applied to the training data set, and the ML model(s) generate an output data set containing predicted melt property values. Training the ML model(s) includes determining an accuracy of the predicted melt property values based one comparing the predicted melt property values to the measured melt property values of the training data set. After determining accuracy of the ML models, the melt property prediction computer 140 can be configured to adjust the ML model(s).

Training with the training data set can be repeated until the accuracy of the predicted melt property values is within a desired accuracy, e.g., predicted melt property values are within +/−5%, 4%, 3%, 2%, 1%, 0.9%, 0.8%, 0.7%, 0.6%, 0.5%, 0.4%, 0.3%, 0.2%, 0.1% of the corresponding measured melt property values of the training data set.

In some aspects, training the ML model(s) can include scaling the measured melt property value(s) from the training data set. For example, the measured melt property values can be scaled to be a value in the range of 0 to 1, −1 to 1, or any other range suitable for scaling based on a resin grade of the respective sample. The resin grade can be selected from a list of grades where each resin grade defines properties of the polymer fluff, such as the density (high density polyethylene (HDPE), medium density polyethylene (MDPE), low density polyethylene (LDPE), linear low density polyethylene (LLDPE), or metallocene polyethylene) and/or end use application (blow molding, injection molding, pipe and corrugated extrusion, rotational molding, sheet extrusion, blown film, cast film, or extraction coating/lamination). Scaling can be performed by the melt property prediction computer 140. The melt property prediction computer 140 can receive or retrieve the measured melt property value from the database 130, and the melt property prediction computer 140 can then scale the measured melt property values. Alternatively, the melt property prediction computer 140 can receive the measured melt property values via input from a technician or other operator. The melt property prediction computer 140 can then scale the entered measured melt property values.

At block 330, the method 300 includes applying the trained ML model(s) to an input data set to output a predicted melt property value for a polymer extrudate, where the polymer extrudate at block 330 is not the polymer extrudate that is produced at block 310. That is the polymer extrudate made at block 330 is made at a different time than the polymer extrudate made at block 310.

In aspects, the method 300 can include constructing the input data set prior to performing block 330. The input data set can be constructed by the melt property prediction computer 140. Construction of the input data set can include any technique for building a feature set for a machine learning model that is appropriate for feeding the input data set to the machine learning model disclosed herein.

In some aspects, the input data set can include average value data points for the operating parameters of the polymer extruder 110 that are generated while extruding the polymer extrudate. In aspects, each of the average value data points is an average value for a time-series data set for one of the operating parameters of the polymer extruder 110 over an interval of time. In some aspects, the number of operating parameters can include about 30 features or fewer, such as about 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 operating parameters. In some aspects selection of the operating parameters is based on the reliability of the instrumentation associated with one or more of the operating parameters and the impact the operating parameters has on the accuracy of the predictions. It has been discovered that reducing the number of operating parameters in the input data set from about 100 or more operating parameters to about 10 to about 30 operating parameters reduces error rates of the predictions. The operating parameters and associated delta values can also be referred to as features.

The input data set includes an average value data point or raw value data point for each of the following operating parameters of the polymer extruder 110:
i) counts measured in the master feed line 102b,
ii) the extruder fluff feed rate measured by the flow meter in inlet 205,
iii) a speed of the drive motor 210,
iv) one or more temperatures in one or more zones of the screw portion 220,
v) one or more temperatures of the polymer (e.g., polymer melt or molten polymer) in one or more zones of the screw portion 220),
vi) a pressure in one or more zones of the screw portion 220,
vii) one or more temperatures in one or more zones of the melt flow portion 240,
viii) a temperature for at least one bearing (or each bearing) of the gear pump 230,
ix) a temperature of the oil of the gear pump 230 (e.g., oil temperature of a finishing gear),
x) an amperage of the gear pump 230,
xi) a speed of the gear pump 230,
xii) suction pressure of the gear pump 230,
xiii) a discharge pressure of the gear pump 230,
xiv) a screenpack 250a differential pressure,
xv) a temperature of the die plate 250c,
xvi) a pressure in the die plate 250c,
xvii) a speed of the pelletizer 260,
xviii) a temperature of the polymer in the die plate 250c, or
xix) combinations thereof.

In additional or alternative aspects, the input data set can alternatively include the type of the polymer (e.g., homopolymer, copolymer, polyethylene, ethylene-butene-copolymer, etc.), an anchor melt property value which is the most-recent measured melt property value of a sample of the polymer that is being extruded, a value for the interval of time that elapsed since the most-recent sample was collected, an identifier for the polymer extruder 110, an average value or raw value for each of the following operating parameters:
i) the counts measured in the master feed line 102b,
ii) the extruder fluff feed rate measured by the flow meter in inlet 205,
iii) a speed of the drive motor 210,
iv) one or more temperatures in one or more zones of the screw portion 220,
v) one or more temperatures of the polymer (e.g., polymer melt or molten polymer) in one or more zones of the screw portion 220),
vi) a pressure in one or more zones of the screw portion 220,
vii) one or more temperatures in one or more zones of the melt flow portion 240,
viii) a temperature for at least one bearing (or each bearing) of the gear pump 230,
ix) a temperature of the oil of the gear pump 230 (e.g., oil temperature of a finishing gear),
x) an amperage of the gear pump 230,
xi) a speed of the gear pump 230,
xii) a suction pressure of the gear pump 230,
xiii) a discharge pressure of the gear pump 230,
xiv) a screenpack 250a differential pressure,
xv) a temperature of the die plate 250c,
xvi) a pressure in the die plate 250c,
xvii) a speed of the pelletizer 260,
xviii) a temperature of the polymer in the die plate 250c,
xix) a polymerization reactor polymer production rate,
xx) a ratio of power to amperage of the gear pump 230, or
xxi) combinations thereof, and a change in value or “delta value” for the each of the above operating parameters over the interval of time. In some aspects, examples of the delta values can include:
i) a delta value for the pressure in the die plate 250c,
ii) a delta value for the temperature of the polymer in the die plate 250c,
iii) a delta value for the speed of the drive motor 210,
iv) a delta value for the extruder fluff feed rate,
v) a delta value for the temperature of the oil of the gear pump 230 (e.g., oil temperature of a finishing gear),
vi) a delta value for the temperature for at least one bearing (or each bearing) of the gear pump 230,
vii) a delta value for the amperage of the gear pump 230,
viii) a delta value for the discharge pressure of the gear pump 230,
ix) a delta value for the suction pressure of the gear pump 230,
x) a delta value for the speed of the gear pump 230,
xi) a delta value for the ratio of power to amperage of the gear pump 230,
xii) a delta value for the counts measured in the master feed line 102b,
xiii) a delta value for the speed of the pelletizer 260,
xiv) a delta value for the polymerization reactor polymer production rate,
xv) a delta value for the screenpack 250a differential pressure
xvi) a delta value for one or more temperatures in one or more zones of the screw portion 220,
xvii) a delta value for one or more temperatures of the polymer (e.g., polymer melt or molten polymer) in one or more zones of the screw portion 220),
xviii) a delta value for the pressure in one or more zones of the screw portion 220, or
xix) combinations thereof.

In aspects of this disclosure, polymerization reactor polymer production rate can be calculated for use in the input data set. For example, in a gas phase or loop slurry polymerization system, polymer is produced by exothermic reaction of olefin monomers in presence of a catalyst to form the polymer. The polymerization reactor polymer production rate of polymer can be calculated in time series measurements using equipment and techniques known in the art, and the polymerization reactor polymer production rate at points in time or averaged over an interval of time of the polymer fluff can be used as the polymerization reactor polymer production rate that is associated with the extruder operating parameters, e.g., according to an association such as having the same point in time or interval of time. For example, polymerization reactor polymer production rate of polymer can be calculated by a mass balance across the reactor (mass of reactor feed products in—mass of liquid & vapor hydrocarbons out) or by an energy balance that calculates the amount of heat removed from the reactor. In aspects, each calculated polymerization reactor production rate is associated with a point in time, where the point in time is the actual time point when the polymer was made by the reactor plus an amount of time that accounts for the time it takes for polymer to flow from the reactor to the extruder.

In aspects, the anchor melt property value associated with the sample can be received by the melt property prediction computer 140 from the rheometer 150, e.g., in real-time, as the most-recent measured melt property value 155 to be used as the anchor melt property value in the input data set by the melt property prediction computer 140. In alternative aspects, an operator personnel can view the measured melt property value and enter the measured melt property value into the melt property prediction computer 140 via an interface for entering data such as display, keyboard, and data input software.

Block 330 can provide real-time melt property prediction of polymer extrudate produced by a polymer extruder 110 by applying the trained ML model(s) to an input data set comprising operating parameter values of the polymer extruder 110 that are received in real-time from the polymer extruder 110, the extruder data computer system 120, the database 130, or combinations thereof.

In aspects, the polymer extrudate made at block 330 is extruded in the polymer extruder 110 during this interval of time discussed for block 330. In aspects, the interval of time discussed for block 330 is equal to the interval of time discussed for block 310. The time-series data set is generated from time-series real-time data received from the database 130 or directly from the extruder data computer system 120, for production of a polymer extrudate in the extruder 110 during an interval of time discussed for block 330 that is after completion of training of the ML model(s).

In embodiments where the ML model(s) is trained using measured melt property values that are scaled, the predicted melt property values that are output can be scaled on the same scale as the measured melt property values. It was found that scaling improved accuracy of the predicted melt property values. For scaling with an ML model called XGBoost (described above), it was found that scaling improved accuracy of the predicted melt property values by 10-15%, even though the XGBoost model recommend that the input data is not scaled. Thus, scaling with XGBoost unexpectedly provided more accurate predicted melt property values, since the XGBoost model recommends no scaling.

In embodiments, the predicted melt property values can be displayed on a display of the melt property prediction computer 140 in a manner that is observable by a technician or other operator of the extruder 110, such as a real-time graph of predicted melt property value versus time. In aspects, block 330 is performed after block 310.

At decision block 340, the method 300 can include determining whether the output data set containing the predicted melt property values needs post-processing. Determining “NO” ends the method 300 in FIG. 3. Determining “YES” results in the method 300 moving to block 350.

At block 350, the method 300 can include processing the predicted melt property values contained in the output data set. In aspects, processing can include unscaling predicted melt property values to create predicted unscaled melt property values. Embodiments of the disclosure contemplate that ML model(s) may be trained using scaled (e.g., on a scale of 0 to 1, or on a scale of −1 to 1 based on a resin grade of the sample) measured melt property values, and thus, the trained ML model(s) can be trained to output predicted melt property values based on the scale by which the ML model(s) were trained. The predicted melt property values can be unscaled, and the predicted unscaled melt property values can be displayed on a display of the melt property prediction computer 140 in a manner that is observable by a technician or other operator of the extruder 110, such as a real-time graph of predicted unscaled melt property value versus time.

In aspects, the method 300 can additionally include operating the polymer extruder 110 to form the polymer extrudate; and generating time-series real-time extruder data during the operating. In aspects, the time-series real-time extruder data corresponds to the operating parameters of the polymer extruder 110 while forming the polymer extrudate (e.g., at a given point in time). The polymer extruder 110 can be operated according to any technique discussed herein, and operation of the polymer extruder 110 generates the time-series real-time extruder data, which is sent from the polymer extruder 110 to the extruder data computer system 120. The time-series real-time extruder data is sent to database 130 as described herein, and in aspects, the method 300 can also include receiving or retrieving, by the melt property prediction computer 140, the time-series real-time extruder data. In these aspects, the method 300 can additionally include constructing the input data set after receiving or retrieving, according to a technique described herein.

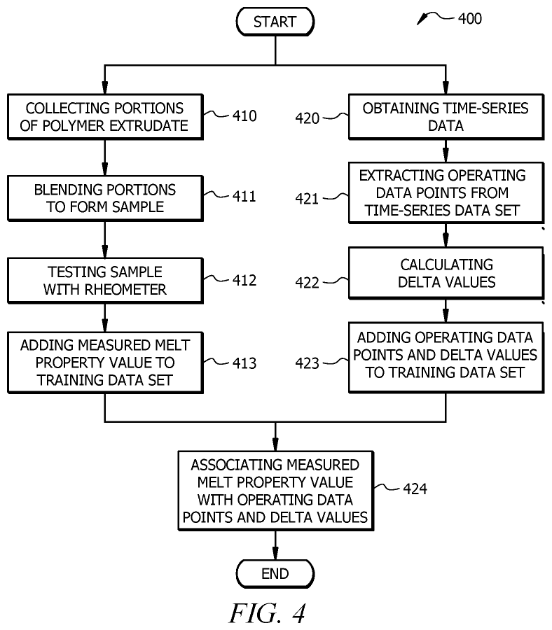

FIG. 4 illustrates a flow diagram of a method 400 for obtaining a training data set used to train a machine learning model according to the disclosure. The method 400 is an embodiment for obtaining a training data set that can be performed as block 310 of the method 300 in FIG. 3. It is contemplated that the various components of the polymer extrusion system 100 of FIG. 1 are used to perform the method 400, and steps of the method 400 are described with reference to components of the polymer extrusion system 100 in FIG. 1.

Performing blocks 410 to 413 of the method 400 generates a historical measured melt property value corresponding to a sample of a polymer extrudate previously obtained from the polymer extruder 110. The measured melt property value is a historical value based on a sample made at a past point in time or during a past interval of time. In aspects, it is contemplated that blocks 410, 411, and 412 can be performed for any previously produced polymer extrudate that was not tested for a melt property value. Alternatively, aspects of the method 400 contemplate that blocks 410, 411, and 412 were already performed for a previously produced polymer extrudate, and the method 400 can include only block 413 of those blocks 410 to 413 shown in FIG. 4, without performing blocks 410, 411, and 412.

Performing blocks 420 to 423 of the method 400 generates historical operating data points for an operating parameter of the polymer extruder 110. Blocks 420 to 423 can be repeated to obtain operating data points for additional operating parameters of the polymer extruder 110 corresponding to the historical measured melt property value obtained in blocks 410 to 413.

Repeating the method 400 generates i) a plurality of historical measured melt property values corresponding to a plurality of samples of one or more polymer extrudates historically obtained from the polymer extruder 110 ii) a first plurality of operating data points for a plurality of operating parameters of the polymer extruder 110 that form at least part of the training data set, and iii) a first plurality of delta values corresponding to a difference between the first plurality of operating data points and a second plurality of operating data points of the polymer extruder 110.

In aspects, for any historical sample that was collected over an interval of time, the operating data points can be average values derived from the time-series data set for each of the plurality of operating parameters generated over the interval of time, where the average value is based on the interval of time (e.g., calculated as the average of each data point value that is present in the time-series data over the interval of time).

In alternative aspects, for any historical sample that was collected at a point in time, the operating data points can be raw data values derived from the time-series data set for each of the plurality of operating parameters generated at the point in time.

In aspects, the operating data points in a training data set can include both average values for those samples collected over a respective interval of time for the sample and raw data values for those samples collected at their respective point in time.

It should be appreciated that blocks 410 to 413 can be performed at points in time that are different than performance of blocks 420 to 423. For example, the obtaining the training data set can include i) obtaining measured melt property values for a past time period, such as the previous 10 years, and ii) obtaining the time-series data corresponding to when the samples were made in the previous 10 years and the associated measured melt property values for the samples. In this example, blocks 410 to 412 can have been performed around the time when the historical polymer extrudate was produced at some time in the past, and blocks 420 to 423 can be performed at some later time when the training data set is constructed or obtained. In some aspects, the method 400 can include retrieving or looking up the measured melt property values that were generated and recorded in the past. Using historical melt property values and the associated historical time series data in method 400 can increase the size of the data set, which can improve the training of the ML model(s) and make predictions of melt property values more accurate.

It should also be appreciated that blocks 410 and 420 can be performed concurrently to generate melt property values and time-series data that are used to obtain the training data set in the method 400. That is, blocks 410 and 420 can involve operating the polymer extruder 110 such that portions of polymer extrudate are collected from the polymer extruder 110 in block 410 and time-series data is generated by the polymer extruder 110 and received by the extruder data computer system 120 in block 420. That is, the time-series data generated and received in block 420 is for the manufactured polymer extrudate from which portions of polymer extrudate are collected in block 410.

At block 410, the method 400 can include collecting portions of polymer extrudate. The portions of the polymer extrudate are collected from the polymer extruder 110, e.g., from the pelletizer 260 of the polymer extruder 110. A continuous flow of polymer extrudate can move from the pelletizer 260 to a transfer line 106. A “portion” of the polymer extrudate can refer to a fraction of the total flow of the polymer extrudate from the pelletizer 260. Portion of the polymer extrudate in the flow can be collected continuously or periodically. For example, a portion of the polymer extrudate that continuously flows from the pelletizer 260 may be collected continuously by directing the flow of polymer extrudate over a screen or a hole in a tray or conduit through which only a portion of the polymer extrudate can pass as the mass of polymer extruder flows over the tray or through the conduit. In another example, a portion of the polymer extrudate continuously flows from the pelletizer 260 may be collected periodically by periodically actuating a valve in a line (e.g., in line 108) that allows polymer extrudate to flow into a sample collection container for a brief period of time, such as for 1, 2, 3, 4, or 5 seconds.

In embodiments, the portions can be collected for an interval of time that is a sample collection frequency, in that, a sample is formed by the portions collected after the occurrence of the interval of time, repeated at the sample collection frequency.

At block 411, the method 400 can include blending the portions of the polymer extrudate that were collected at block 410 to form a sample. A “sample” of polymer extrudate as disclosed herein can include the portions of polymer extrudate collected over the interval of time that is referred to herein as the sample collection frequency. For example, a “sample” can include one hundred twenty portions of polymer extrudate that were collected every one minute for two hours (e.g., one hundred twenty portions because one portion was collected every minute for one hundred twenty minutes). Blending can be accomplished by warming the portions of polymer extrudate and evenly mixing the warmed or melted polymer extrudate into a blended extrudate.

At block 412, the method 400 can include testing the sample with the rheometer 150 to obtain a measured melt property value, such as a measured melt flow value (MF value), a measured melt index value (MI₂ value or MI₅ value), or a measured high load melt index value (HLMI value). Testing the sample can be performed by the rheometer 150.

At block 413, the method 400 can include adding the measured melt property value to the training data set. The measured melt property value that is added to the training data set can be scaled or unscaled. It is contemplated that a training data set contains only scaled measured melt property values or unscaled measured melt property values, but not both.

In embodiments, the measured melt property value that is added to the training data set can be unscaled. The unscaled measured melt property value can be added to the training data set by the melt property prediction computer 140. In some aspects, the melt property prediction computer 140 can receive/retrieve the measured melt property value from the database 130; alternatively, the rheometer 150 can be networked with the melt property prediction computer 140, and the rheometer 150 can be configured to send the measured melt property value to the melt property prediction computer 140, and the melt property prediction computer 140 can be configured to receive the measured melt property value and add the measured melt property value to the training data set; alternatively, the measured melt property value can be added to the training data set by a technician or other operator, for example, via an interface such as mouse and keyboard or touchscreen of the melt property prediction computer 140. The melt property prediction computer 140 can then add the entered measured melt property value to the training data set.

Blocks 410 to 413 of the method 400 can be repeated any number of times to increase the number of melt property values associated with samples in the training data set. In aspects, repeating blocks 410 to 413 can occur at the sample collection frequency. At block 420, the method 400 can include obtaining time-series data. The time-series data can be obtained, for example, by the melt property prediction computer 140 from the extruder data computer system 120 or the database 130. The time-series data includes operating parameter values for the polymer extruder 110 for the same interval(s) of time and/or point(s) in time the portions of polymer extrudate are collected to form the sample in blocks 410 to 411. The operating parameter values can correspond to any combination of the sensor signals in FIG. 2.

At block 421, the method 400 can include extracting operating data points from the time-series data. The operating data points include operating parameter values corresponding to the portions of polymer extrudate collected at the respective interval(s) of time and/or point(s) in time.

At block 422, the method 400 can include calculating delta values. The delta values can be calculated by the melt property prediction computer 140. Each of the delta values corresponds to a difference between i) the operating data points at the point in time when the corresponding first sample was collected and ii) the operating data points at the point in time (or endpoint of another interval of time) when another second sample was collected. In aspects, the other second sample can be a previous sample that was collected from the polymer extruder before the first sample was collected; alternatively, the other second sample can be a sample that was collected after the first sample was collected.

In aspects where a corresponding sample was collected over an interval of time, the method 400 at block 422 can also include calculating average values for the operating data points over the interval of time. As discussed above, the average value is based on the interval of time (e.g., calculated as the average of each data point value that is present in the time-series data over the interval of time).

At block 423, the method 400 can include adding the operating data points (raw data values, average values, or a combination of raw data values and average values) and the delta values to the training data set. The operating data points and delta values can be added to the training data set by the melt property prediction computer 140.

At block 424, the method 400 can include associating the historical measured melt property value that is added to the training data set with the historical operating data points and historical delta values that are added to the training data set. The melt property prediction computer 140 can perform the association. In aspects, the association can be based on the interval of time or point in time corresponding to the measured melt property value and corresponding to the operating data points and delta values. In embodiments, associating can include labeling the historical measured melt property value that is added to the training data set and the historical operating data points and delta values that are added to the training data set with the interval of time or point in time. Labeling may be part of a supervised training of a machine learning model disclosed herein.

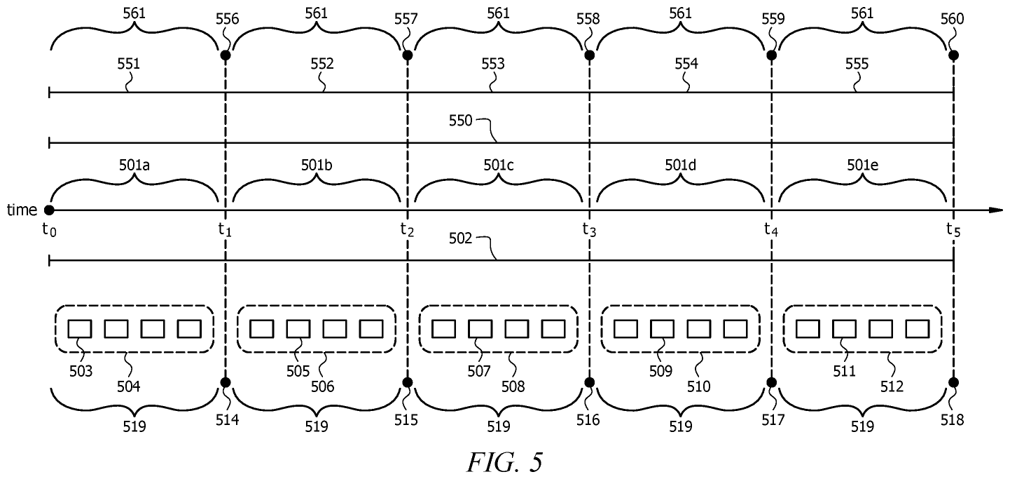

FIG. 5 is a schematic diagram illustrating how average values can be calculated when samples are made by combining portions of polymer extrudate that are collected over a sample collection frequency.

The time axis has five intervals of time illustrated: interval of time 501a that is from t₀ to t₁, interval of time 501b that is from t₁ to t₂, interval of time 501c that is from t₂ to t₃, interval of time 501d that is from t₃ to t₄, and interval of time 501e that is from t₄ to t₅. Any combination of intervals of time 501a, 501b, 501c, 501d, 501e can be the same duration; alternatively, none of the intervals of time 501a, 501b, 501c, 501d, 501e have the same duration.

Polymer extrudate 502 is produced by the polymer extruder 110 from time t₀ to time t₅. Portions 503 of polymer extrudate 502 are collected over interval of time 501a that is from t₀ to t₁, and blended into sample 504. Portions 505 of polymer extrudate 502 are collected over interval of time 501b that is from t₁ to t₂, and blended into sample 506. Portions 507 of polymer extrudate 502 are collected over interval of time 501c that is from t₂ to t₃, and blended into sample 508. Portions 509 of polymer extrudate 502 are collected over interval of time 501d that is from t₃ to t₄, and blended into sample 510. Portions 511 of polymer extrudate 502 are collected over interval of time 501e that is from t₄ to t₅, and blended into sample 512. These actions can be performed in blocks 410 and 411 of the method 400.

The samples 504, 506, 508, 510, and 512 are tested with rheometer 150 to obtain a corresponding measured melt property value. Testing sample 504 produces measured melt property value 514. Testing sample 506 produces measured melt property value 515. Testing sample 508 produces measured melt property value 516. Testing sample 510 produces measured melt property value 517. Testing sample 512 produces measured melt property value 518.

The portions for each sample are collected over the interval of time 501a to 501e which are each equal to the sample collection frequency 519. Moreover, each measured melt property value 514 to 518 corresponds to the polymer extrudate collected over the sample collection frequency 519. As discussed herein, each measured melt property value 514 to 518 may be scaled. The measured melt property values 514 to 518 are added to the training data set.

Because each sample 504, 506, 508, 510, and 512 is collected over a respective interval of time 501a, 501b, 501c, 501d, 501e, the operating data points are average value data points calculated from raw value data points.

Time-series data 550 contains raw value data points for an operating parameter produced by the polymer extruder 110 from time t₀ to time t₅. For simplicity of discussion, time-series data 550 includes raw value data points for a single operating parameter of the polymer extruder 110.

Operating data points 551, 552, 553, 554, and 555 are extracted from the time-series data 550 by the melt property prediction computer 140. Operating data points 551 have raw value data points for the operating parameter that are generated over interval of time 501a. Operating data points 552 have raw value data points for the operating parameter that are generated over interval of time 501b. Operating data points 553 have raw value data points for the operating parameter that re generated over interval of time 501c. Operating data points 554 have raw value data points for the operating parameter that are generated over interval of time 501d. Operating data points 555 have raw value data points for the operating parameter that are generated over interval of time 501e.

The raw value data points in operating data points 551 are averaged over the interval of time 501a that is from t₀ to t₁ by the melt property prediction computer 140 to produce the average value data point 556. The raw value data points in operating data points 552 are averaged over the interval of time 501b that is from t₁ to t₂ by the melt property prediction computer 140 to produce the average value data point 557. The raw value data points in operating data points 553 are averaged over the interval of time 501c that is from t₂ to t₃ by the melt property prediction computer 140 to produce the average value data point 558. The raw value data points in operating data points 554 are averaged over the interval of time 501d that is from t₃ to t₄ by the melt property prediction computer 140 to produce the average value data point 559. The raw value data points in operating data points 555 are averaged over the interval of time 501d that is from t₄ to t₅ by the melt property prediction computer 140 to produce the average value data point 560.

The average value data points 556 to 560 are associated with the measured melt property values 514 to 519 as described for block 424 in the method 400. For example, average value data point 556 can be associated with measure melt property value 514 according to the interval of time 501a that is t₀ to t₁, upon which each data point 556 and value 514 is based; average value data point 557 can be associated with measure melt property value 515 according to the interval of time 501b that is t₁ to t₂, upon which each data point 557 and value 515 is based; average value data point 558 can be associated with measure melt property value 516 according to the interval of time 501c that is t₂ to t₃, upon which each data point 558 and value 516 is based; average value data point 559 can be associated with measure melt property value 517 according to the interval of time 501d that is t₃ to t₄, upon which each data point 559 and value 517 is based; and average value data point 560 can be associated with measure melt property value 518 according to the interval of time 501e that is t₄ to t₅, upon which each data point 560 and value 518 is based.

The average value data points 556 to 560 are determined over the intervals of time 501a to 501e, each interval of time being equal to the calculation frequency 561. In aspects, the calculation frequency is equal to the sample collection frequency, and the “wavelength” of the frequencies (the amount of time that elapses between start and end of the respective frequency) is equal to the interval of time. In embodiments, the interval of time can be greater than 1, 2, 3, 4, 5, 6, 7, 8, 9, or 10 minutes and less than 24, 23, 22, 21, 20, 19, 18, 17, 16, 15, 14, 13, or 12 hours. The interval of time can be any value between any minimum value disclosed herein and any maximum value disclosed herein.

It has been unexpectedly found that having a calculation frequency of a smaller interval of time than the interval of time used for the sample collection resulted in a break-down of the ML model prediction. That is, it was thought that having more frequent average value data points would lead to more accurate predicted melt property values, and in practice, this was not true. Training the ML model with matched and equal sample collection frequency and calculation frequency unexpectedly resulted in the more accurate predicted melt property values.

In FIG. 5, the sample collection frequency 519 is the same amount of time as (is equal to) the calculation frequency 561. Matching the sample collection frequency 519 with the calculation frequency 561 has been found to produce more accurate melt property value predictions.

### EXAMPLES

An XGBoost machine learning model was trained with a training data set and applied to various input data sets.

The XGBoost machine learning model was trained with a training data set that included historical measured melt property values of various historically collected polymer samples, operating data points for the operating parameters of the polymer extruder that were generated when the samples were collected, and delta values corresponding to the difference between adjacent (relative to a set interval of time). The input data set included these combinations of data for multiple polymer resins generated over a 50 month historical period.

The trained XGBoost machine learning model was then applied to an input data set containing, for every time point in the input data set, the type of polymer (e.g., HDPE homopolymer, HDPE ethylene-hexene copolymer, MDPE homopolymer, MDPE ethylene-hexene copolymer, LLDPE homopolymer, or LLDPE ethylene-hexene copolymer), an anchor melt property value which is the most-recent measured melt property value of a sample of the polymer that was in the simulated melt property data set, a value for the interval of time that elapsed since the most-recent sample was collected, an identifier for the polymer extruder, an average value or raw value for each of the following operating parameters:
i) the counts measured in the master feed line of the extruder,
ii) the extruder fluff feed rate measured by the flow meter in inlet,
iii) a speed of the drive motor,
iv) one or more temperatures in one or more zones of the screw portion,
v) one or more temperatures of the polymer in zones 1 to 10 of the extruder,
vi) a pressure in zone 5 of the extruder,
vii) a temperature in the melt flow portion of the extruder,
viii) an average temperature of the bearings of the gear pump,
ix) an oil temperature of a finishing gear of the gear pump,
x) an amperage of the gear pump,
xi) a speed of the gear pump,
xii) suction pressure of the gear pump,
xiii) a discharge pressure of the gear pump,
xiv) a screenpack differential pressure,
xv) a temperature of the die plate,
xvi) a pressure in the die plate,
xvii) a speed of the pelletizer,
xviii) a temperature of the polymer in the die plate,
xix) polymerization reactor polymer production rate, and
xx) a ratio of power to amperage of the gear pump.
   When delta values were utilized, the following delta values were used:
i) a delta value for the pressure in the die plate,
ii) a delta value for the temperature of the polymer in the die plate,
iii) a delta value for the speed of the drive motor,
iv) a delta value for the extruder fluff feed rate,
v) a delta value for the oil temperature of the first, second, third, fourth, and finishing gears of the gear pump,
vi) a delta value for the gear bearing stable temperature of the gear pump,
vii) a delta value for the amperage of the gear pump,
viii) a delta value for the discharge pressure of the gear pump,
ix) a delta value for the suction pressure of the gear pump,
x) a delta value for the speed of the gear pump,
xi) a delta value for the ratio of power to amperage of the gear pump,
xii) a delta value for the counts measured in the master feed line,
xiii) a delta value for the speed of the pelletizer,
xiv) a delta value for the polymerization reactor polymer production rate,
xv) a delta value for the screenpack differential pressure,
xvi) a delta value for temperatures in zones 1, 2, 3, 4, 5, 6, 7, 8, and 10 of the extruder,
xvii) a delta value for the temperature of the polymer melt or molten polymer in zone 5 of the extruder, and
xviii) a delta value for the pressure in zone 5 of the extruder.

In all Examples 1 to 4, the XGBoost machine learning model was trained with the same features as the input data set, using historical melt property values and associated historical operating data points of a polymer extruder that made the polymer extrudate from which the samples were collected. Example 1 did not use delta values in the training data set and the input data set. Examples 2, 3, and 4 used delta values in the training data set and the input data set.

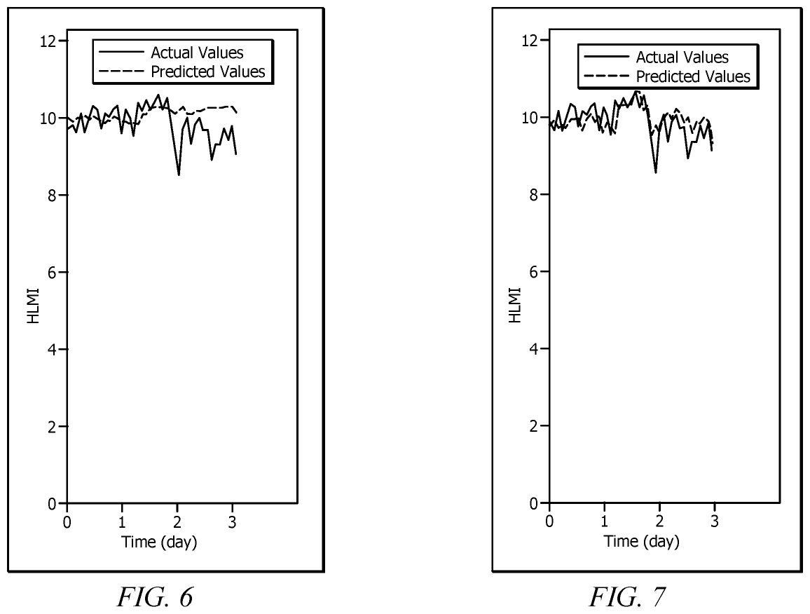

Also in all Examples 1 to 4, the melt property values illustrated in FIGS. 6, 7, 8, and 9 were predicted for known historical melt property values, to ascertain accuracy of the model. That is, the trained XGBoost machine learning model was run on historical time-series data to simulate an online real-time experience in predicting melt property values for the known historical melt property values. The known historical melt property values are illustrated in FIGS. 6, 7, 8, and 9 as the solid lines, and the predicted melt property values are illustrated as the dashed lines. The input data set to generate predicted melt property values included anchor melt property values. The trained XGBoost machine learning model output scaled predicted melt property values, which were unscaled to obtain the unscaled predicted melt property values illustrated in FIGS. 6, 7, 8, and 9.

Example 1 used no delta values to predict melt property values with the trained XGBoost machine learning model. FIG. 6 is a graph of the HLMI value versus time showing a solid line for actual values and a dashed line for values predicted with a machine learning model, where delta values were not used in the input data set.

Example 2 used delta values to predict melt property values with the trained XGBoost machine learning model. FIG. 7 is a graph of the HLMI value versus time showing a solid line for actual values and a dashed line for values predicted with a machine learning model, where delta values were used in the input data set.

Comparing FIG. 6 and FIG. 7, it can be seen that the predicted melt property values are closer to the actual melt property values when delta values are utilized. It has been found that when the training data set, the input data set, or both the training data set and the input data set does not include the change in value or “delta value” of the operating parameters of the polymer extruder 110, a mean absolute percentage error (MAPE) of the predicted melt property values at an upper specification limit and a lower specification limit for the melt property can be as high as 20%. It has been further found that when the training data set does include the change in value or “delta value” of the operating parameters of the polymer extruder 110, a mean absolute percentage error (MAPE) of the predicted melt property values at an upper specification limit and a lower specification limit for the melt property can be less than 8%.

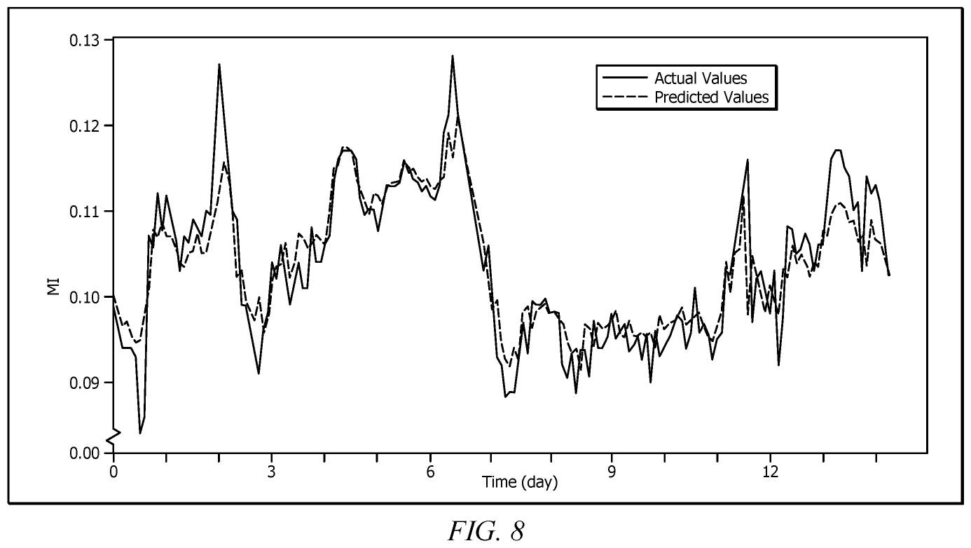

Example 3 used delta values to predict melt property values with the trained XGBoost machine learning model. 2 hours was used as the interval of time to determine delta values. FIG. 8 is a graph of the MI value versus time showing a solid line for actual values and a dashed line for values predicted with a machine learning model, where the delta values were based on 2 hours between the data points for purposes of calculating the delta values.

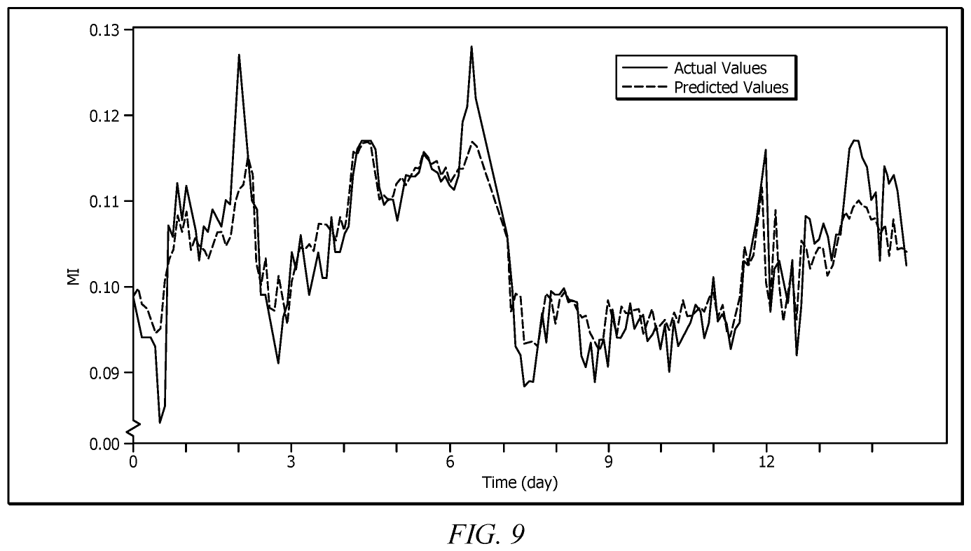

Example 4 used delta values to predict melt property values with the trained XGBoost machine learning model. 4 hours was used as the interval of time to determine delta values. FIG. 9 is a graph of the MI value versus time showing a solid line for actual values and a dashed line for values predicted with a machine learning model, where the delta values were based on 4 hours between the data points for purposes of calculating the delta values.

Comparing FIG. 8 and FIG. 9, it can be seen that the predicted melt property values are closer to the actual melt property values when the delta values are based on 2 hours compared to delta values that are based on 4 hours.

### ADDITIONAL DESCRIPTION

Methods and computers for melt property value prediction have been described. The present application is also directed to the subject-matter described in the following numbered paragraphs (referred to as “para” or “paras”):
Para 1. A method comprising: training a machine learning model with a training data set; wherein the training data set comprises:
   1) i) a first plurality of average value data points for a plurality of operating parameters of a polymer extruder; and ii) a plurality of measured melt property values corresponding to a plurality of samples of a polymer extrudate obtained from the polymer extruder; wherein each of the first plurality of average value data points is an average value for a time-series data set for one of the plurality of operating parameters, wherein the time-series data set is collected over a first interval of time, wherein the first plurality of average value data points includes a plurality of average values determined at a calculation frequency equal to the first interval of time; wherein the calculation frequency is equal to a sample collection frequency for each of the plurality of samples; and wherein the first plurality of average value data points corresponds to time-series data for the plurality of operating parameters of the polymer extruder that is generated while the polymer extruder forms the polymer extrudate; or
   2) i) a measured melt property value for the sample; ii) a first plurality of operating data points for a plurality of operating parameters of the polymer extruder corresponding to when the sample was collected; and iii) a first plurality of delta values corresponding to a difference between the first plurality of operating data points and a second plurality of operating data points of the polymer extruder, wherein the second plurality of operating data points corresponds to a previous sample that was collected from the polymer extruder before the sample was collected.
Para 2: The method of Para 1, further comprising: applying the machine learning model to an input data set to output a predicted melt property value for a second polymer extrudate, wherein the input data set comprises a second plurality of average value data points for the plurality of operating parameters of the polymer extruder.
Para 3: The method of Para 2, wherein each of the second plurality of average value data points is a second average value for a second time-series data set for one of the plurality of operating parameters of the polymer extruder over a second interval of time, wherein the second interval of time is equal to the first interval of time.
Para 4: The method of Para 2 or 3, wherein the second polymer extrudate is extruded in the polymer extruder during the second interval of time.
Para 5: The method of any one of Paras 1 to 5, wherein the plurality of operating parameters comprises i) counts measured in a master feed line of the polymer extruder, ii) a fluff feed rate, iii) a speed of a drive motor of the polymer extruder, iv) one or more temperatures in one or more zones of a screw portion of the polymer extruder, v) one or more temperatures of polymer in the one or more zones of the screw portion, vi) a pressure in one or more zones of the screw portion, vii) one or more temperatures in one or more zones of a molten flow portion of the polymer extruder, viii) a temperature for at least one bearing (or each bearing) of a gear pump, ix) a temperature of the oil of the gear pump (e.g., oil temperature of a finishing gear), x) an amperage of the gear pump, xi) a speed of the gear pump, xii) a discharge pressure of the gear pump, xiii) a differential pressure of a screenpack of a die plate assembly of the polymer extruder, xiv) a temperature of a die plate the die plate assembly, xv) a temperature of polymer in the die plate, xvi) a pressure in the die plate, xvii) a speed of a pelletizer of the polymer extruder, xviii) master feeder counts, or xix) combinations thereof.
Para 6: The method of any one of Paras 1 to 5, further comprising: scaling the plurality of measured melt property values.
Para 7: The method of Para 6, wherein the plurality of measured melt property values are scaled on a scale of 0 to 1 or −1 to 1 based on a resin grade of the sample.
Para 8: The method of any one of Paras 1 to 7, further comprising: applying the machine learning model to an input data set to output a scaled predicted melt property value for a second polymer extrudate, wherein the input data set comprises a second plurality of average value data points for the plurality of operating parameters of the polymer extruder; and unscaling the scaled predicted melt property value to produce a predicted unscaled melt property value.
Para 9: The method of any one of Paras 1 to 8, wherein the machine learning model is supervised.
Para 10: The method of any one of Paras 1 to 9, wherein the machine learning model is a gradient-boosting decision tree model.
Para 11: The method of any one of Paras 1 to 10, wherein the polymer extrudate is a homopolymer or copolymer of one or more olefin monomers.
Para 12: The method of any one of Paras 1 to 12, wherein the plurality of measured melt property values are MF values, MI₂ values, MI₅ values, or HLMI values.
Para 13: A melt property value prediction computer having one or more processors and a memory having instructions stored thereon that cause the one or more processors to: train a machine learning model with a training data set; wherein the training data set comprises: i) a first plurality of average value data points for a plurality of operating parameters of a polymer extruder; and ii) a plurality of measured melt property values corresponding to a plurality of samples of a polymer extrudate obtained from the polymer extruder; wherein each of the first plurality of average value data points is an average value for a time-series data set for one of the plurality of operating parameters, wherein the time-series data set is collected over a first interval of time, wherein the first plurality of average value data points includes a plurality of average values determined at a calculation frequency equal to the first interval of time; wherein the calculation frequency is equal to a sample collection frequency for each of the plurality of samples; and wherein the first plurality of average value data points corresponds to time-series data for the plurality of operating parameters of the polymer extruder that is generated while the polymer extruder forms the polymer extrudate.
Para 14: The melt property value prediction computer of Para 13, wherein the instructions further cause the one or more processors to: apply the machine learning model to an input data set to output a predicted melt property value for a second polymer extrudate, wherein the input data set comprises a second plurality of average value data points for the plurality of operating parameters of the polymer extruder.
Para 15: The melt property value prediction computer of Para 14, wherein each of the second plurality of average value data points is a second average value for a second time-series data set for one of the plurality of operating parameters of the polymer extruder over a second interval of time, wherein the second interval of time is equal to the first interval of time.
Para 16: The melt property value prediction computer of Para 14 or 15, wherein the plurality of measured melt property values are scaled values of a plurality of melt property test results.
Para 17: The melt property value prediction computer of Para 16, wherein the instructions further cause the one or more processors to: unscale the predicted melt property value to create a predicted unscaled melt property value.
Para 18: The melt property value prediction computer of any one of Paras 13 to 17, wherein the machine learning model is a decision tree-based model.
Para 19: The melt property value prediction computer of any one of Paras 13 to 18, wherein the plurality of measured melt property values are MF values, MI₂ values, MI₅ values, or HLMI values.
Para 20: The melt property value prediction computer any one of Paras 13 to 19, wherein the plurality of operating parameters comprises i) counts measured in a master feed line of the polymer extruder, ii) a fluff feed rate, iii) a speed of a drive motor of the polymer extruder, iv) one or more temperatures in one or more zones of a screw portion of the polymer extruder, v) one or more temperatures of polymer in the one or more zones of the screw portion, vi) a pressure in one or more zones of the screw portion, vii) one or more temperatures in one or more zones of a melt flow portion of the polymer extruder, viii) a temperature for at least one bearing (or each bearing) of a gear pump, ix) a temperature of the oil of the gear pump (e.g., oil temperature of a finishing gear), x) an amperage of the gear pump, xi) a speed of the gear pump, xii) a discharge pressure of the gear pump, xiii) a differential pressure of a screenpack of a die plate assembly of the polymer extruder, xiv) a temperature of a die plate the die plate assembly, xv) a temperature of polymer in the die plate, xvi) a pressure in the die plate, xvii) a speed of a pelletizer of the polymer extruder, xviii) master feeder counts, or xix) combinations thereof.
Para 21: A method comprising: applying, while a polymer extruder produces a first polymer extrudate, a machine learning model to an input data set to output a predicted melt property value for the first polymer extrudate, wherein the input data set comprises a raw value data point for each of a plurality of operating parameters of the polymer extruder at a first point in time.
Para 22: The method of Para 21, wherein the input data set further comprises: a delta value for each of the plurality of operating parameters, wherein the delta value is a difference between the raw value data point at the first point in time and a previous raw value data point for each of the plurality of operating parameters of the polymer extruder at a second point in time.
Para 23: The method of Para 21 or 22, wherein the input data set further comprises: a measured melt property value for a sample of a second polymer extrudate obtained before the first point in time.
Para 24: The method of any one of Paras 21 to 23, wherein the measured melt property value is scaled on a scale of −1 to 1 based on a resin grade of the sample.
Para 25: The method of any one of Paras 21 to 24, further comprising: operating the polymer extruder to form the first polymer extrudate; generating time-series real-time extruder data during the operating, wherein the time-series real-time extruder data corresponds to the plurality of operating parameters of the polymer extruder at the first point in time; receiving or retrieving the time-series real-time extruder data; and constructing the input data set after receiving or retrieving.
Para 26. The method of any one of Paras 21 to 25, further comprising: training the machine learning model with a training data set; wherein the training data set comprises, for each sample of polymer extrudate obtained from the polymer extruder: (i) a measured melt property value for the sample; (ii) a first plurality of operating data points for a plurality of operating parameters of the polymer extruder corresponding to when the sample was collected; and (iii) a first plurality of delta values corresponding to a difference between the first plurality of operating data points and a second plurality of operating data points of the polymer extruder, wherein the second plurality of operating data points corresponds to a previous sample that was collected from the polymer extruder before the sample was collected.
Para 27: The method of Para 26, wherein each sample is collected over a first interval of time, wherein each of the first plurality of operating data points is an average value for a time-series data set for one of the plurality of operating parameters collected over the first interval of time, wherein the average value is based on the first interval of time.
Para 28: The method of Para 26, wherein each sample is collected at a respective point in time, wherein each of the first plurality of operating data points is a raw data value for a time-series data set for one of the plurality of operating parameters at the sample's respective point in time.
Para 29: The method of any one of Paras 26 to 28, wherein the measured melt property value is scaled on a scale of −1 to 1 based on a resin grade of the sample.
Para 30: The method of any one of Paras 21 to 29, wherein the plurality of operating parameters comprises i) counts measured in a master feed line of the polymer extruder, ii) a fluff feed rate, iii) a speed of a drive motor of the polymer extruder, iv) one or more temperatures in one or more zones of a screw portion of the polymer extruder, v) one or more temperatures of polymer in the one or more zones of the screw portion, vi) a pressure in one or more zones of the screw portion, vii) one or more temperatures in one or more zones of a molten flow portion of the polymer extruder, viii) a temperature for at least one bearing of a gear pump of the polymer extruder, ix) a temperature of the oil of the gear pump, x) an amperage of the gear pump, xi) a speed of the gear pump, xii) a suction pressure of the gear pump, xiii) a discharge pressure of the gear pump, xiv) a differential pressure of a screenpack of a die plate assembly of the polymer extruder, xv) a temperature of a die plate the die plate assembly, xvi) a temperature of polymer in the die plate, xvii) a pressure in the die plate, xviii) a speed of a pelletizer of the polymer extruder, xix) a ratio of power to amperage of the gear pump, or xx) combinations thereof.
Para 31: The method of any one of Paras 21 to 30, wherein the predicted melt property value is scaled on a scale of 0 to 1 or −1 to 1 based on a resin grade of a polymer fluff that is fed to the polymer extruder to produce the polymer extrudate and/or based on the scale by which the ML model(s) were trained, the method further comprising: unscaling the predicted melt property value to produce a predicted unscaled melt property value.
Para 32: The method of any one of Paras 21 to 31, wherein the machine learning model is supervised.
Para 33: The method of any one of Paras 21 to 32, wherein the machine learning model is a gradient-boosting decision tree model.
Para 34: The method of any one of Paras 21 to 33, wherein the first polymer extrudate is a homopolymer or copolymer of one or more olefin monomers.
Para 35: The method of any one of Paras 21 to 34, wherein measured melt property value is a MF value, a MI2 value, a MI5 value, or a HLMI value.
Para 36: A melt property value prediction computer having one or more processors and a memory having instructions stored thereon that cause the one or more processors to: apply, while a polymer extruder produces a first polymer extrudate, a machine learning model to an input data set to output a predicted melt property value for the first polymer extrudate, wherein the input data set comprises a raw value data point for each of a plurality of operating parameters of the polymer extruder at a first point in time.
Para 37: The melt property value prediction computer of Para 36, wherein the input data set further comprises: a delta value for each of the plurality of operating parameters, wherein the delta value corresponds to a difference between the raw value data point at the first point in time and a previous raw value data point for each of the plurality of operating parameters of the polymer extruder at a second point in time.
Para 38: The melt property value prediction computer of Para 36 or 37, wherein the input data set further comprises: a measured melt property value for a sample of a second polymer extrudate obtained before the first point in time.
Para 39: The melt property value prediction computer of any one of Paras 36 to 38, wherein the instructions further cause the one or more processors to: train the machine learning model with a training data set; wherein the training data set comprises, for each sample of polymer extrudate obtained from the polymer extruder: (i) a measured melt property value for the sample; (ii) a first plurality of operating data points for a plurality of operating parameters of the polymer extruder corresponding to when the sample was collected; and (iii) a first plurality of delta values corresponding to a difference between the first plurality of operating data points and a second plurality of operating data points of the polymer extruder, wherein the second plurality of operating data points corresponds to a previous sample that was collected from the polymer extruder before the sample was collected.
Para 40: The melt property value prediction computer of any one of Paras 36 to 39, wherein the plurality of operating parameters comprises i) counts measured in a master feed line of the polymer extruder, ii) a fluff feed rate, iii) a speed of a drive motor of the polymer extruder, iv) one or more temperatures in one or more zones of a screw portion of the polymer extruder, v) one or more temperatures of polymer in the one or more zones of the screw portion, vi) a pressure in one or more zones of the screw portion, vii) one or more temperatures in one or more zones of a molten flow portion of the polymer extruder, viii) a temperature for at least one bearing of a gear pump of the polymer extruder, ix) a temperature of the oil of the gear pump, x) an amperage of the gear pump, xi) a speed of the gear pump, xii) a suction pressure of the gear pump, xiii) a discharge pressure of the gear pump, xiv) a differential pressure of a screenpack of a die plate assembly of the polymer extruder, xv) a temperature of a die plate the die plate assembly, xvi) a temperature of polymer in the die plate, xvii) a pressure in the die plate, xviii) a speed of a pelletizer of the polymer extruder, xix) a ratio of power to amperage of the gear pump, or xx) combinations thereof.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, methods and steps described in the specification. As one of ordinary skill in the art will readily appreciate from the disclosure, processes, machines, manufacture, compositions of matter, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, methods, or steps.

What is claimed is:

1. A method comprising:
applying, while a polymer extruder produces a first polymer extrudate, a machine learning model to an input data set to output a predicted melt property value for the first polymer extrudate, wherein the input data set comprises a raw value data point for each of a plurality of operating parameters of the polymer extruder at a first point in time.

2. The method of claim 1, wherein the input data set further comprises: a delta value for each of the plurality of operating parameters, wherein the delta value is a difference between the raw value data point at the first point in time and a previous raw value data point for each of the plurality of operating parameters of the polymer extruder at a second point in time.

3. The method of claim 2, wherein the input data set further comprises: a measured melt property value for a sample of a second polymer extrudate obtained before the first point in time.

4. The method of claim 3, wherein the measured melt property value is scaled on a scale of −1 to 1 based on a resin grade of the sample.

5. The method of claim 1, further comprising:
operating the polymer extruder to form the first polymer extrudate;
generating time-series real-time extruder data during the operating, wherein the time-series real-time extruder data corresponds to the plurality of operating parameters of the polymer extruder at the first point in time;
receiving or retrieving the time-series real-time extruder data; and
constructing the input data set after receiving or retrieving.

6. The method of claim 1, further comprising:
training the machine learning model with a training data set;
wherein the training data set comprises, for each sample of polymer extrudate obtained from the polymer extruder:
i) a measured melt property value for the sample;
ii) a first plurality of operating data points for a plurality of operating parameters of the polymer extruder corresponding to when the sample was collected; and
iii) a first plurality of delta values corresponding to a difference between the first plurality of operating data points and a second plurality of operating data points of the polymer extruder, wherein the second plurality of operating data points corresponds to a previous sample that was collected from the polymer extruder before the sample was collected.

7. The method of claim 6, wherein each sample is collected over a first interval of time, wherein each of the first plurality of operating data points is an average value for a time-series data set for one of the plurality of operating parameters collected over the first interval of time, wherein the average value is based on the first interval of time.

8. The method of claim 6, wherein each sample is collected at a point in time, wherein each of the first plurality of operating data points is a raw data value for a time-series data set for one of the plurality of operating parameters at the point in time.

9. The method of claim 6, wherein the measured melt property value is scaled on a scale of −1 to 1 based on a resin grade of the sample.

10. The method of claim 1, wherein the plurality of operating parameters comprises i) counts measured in a master feed line of the polymer extruder, ii) a fluff feed rate, iii) a speed of a drive motor of the polymer extruder, iv) one or more temperatures in one or more zones of a screw portion of the polymer extruder, v) one or more temperatures of polymer in the one or more zones of the screw portion, vi) a pressure in one or more zones of the screw portion, vii) one or more temperatures in one or more zones of a molten flow portion of the polymer extruder, viii) a temperature for at least one bearing of a gear pump of the polymer extruder, ix) a temperature of an oil of the gear pump, x) an amperage of the gear pump, xi) a speed of the gear pump, xii) a suction pressure of the gear pump, xiii) a discharge pressure of the gear pump, xiv) a differential pressure of a screenpack of a die plate assembly of the polymer extruder, xv) a temperature of a die plate the die plate assembly, xvi) a temperature of polymer in the die plate, xvii) a pressure in the die plate, xviii) a speed of a pelletizer of the polymer extruder, xix) a ratio of power to amperage of the gear pump, or xx) combinations thereof.

11. The method of claim 1, wherein the predicted melt property value is scaled on a scale of −1 to 1, the method further comprising:
unscaling the predicted melt property value to produce a predicted unscaled melt property value.

12. The method of claim 1, wherein the machine learning model is supervised.

13. The method of claim 1, wherein the machine learning model is a gradient-boosting decision tree model.

14. The method of claim 1, wherein the first polymer extrudate is a homopolymer or copolymer of one or more olefin monomers.

15. The method of claim 1, wherein measured melt property value is a MF value, a MI₂ value, a MI₅ value, or a HLMI value.

16. A melt property value prediction computer having one or more processors and a memory having instructions stored thereon that cause the one or more processors to:
apply, while a polymer extruder produces a first polymer extrudate, a machine learning model to an input data set to output a predicted melt property value for the first polymer extrudate, wherein the input data set comprises a raw value data point for each of a plurality of operating parameters of the polymer extruder at a first point in time.

17. The melt property value prediction computer of claim 16, wherein the input data set further comprises: a delta value for each of the plurality of operating parameters, wherein the delta value corresponds to a difference between the raw value data point at the first point in time and a previous raw value data point for each of the plurality of operating parameters of the polymer extruder at a second point in time.

18. The melt property value prediction computer of claim 16, wherein the input data set further comprises: a measured melt property value for a sample of a second polymer extrudate obtained before the first point in time.

19. The melt property value prediction computer of claim 16, wherein the instructions further cause the one or more processors to:
train the machine learning model with a training data set;
wherein the training data set comprises, for each sample of polymer extrudate obtained from the polymer extruder:
i) a measured melt property value for the sample;
ii) a first plurality of operating data points for a plurality of operating parameters of the polymer extruder corresponding to when the sample was collected; and
iii) a first plurality of delta values corresponding to a difference between the first plurality of operating data points and a second plurality of operating data points of the polymer extruder, wherein the second plurality of operating data points corresponds to a previous sample that was collected from the polymer extruder before the sample was collected.

20. The melt property value prediction computer of claim 16, wherein the plurality of operating parameters comprises i) counts measured in a master feed line of the polymer extruder, ii) a fluff feed rate, iii) a speed of a drive motor of the polymer extruder, iv) one or more temperatures in one or more zones of a screw portion of the polymer extruder, v) one or more temperatures of polymer in the one or more zones of the screw portion, vi) a pressure in one or more zones of the screw portion, vii) one or more temperatures in one or more zones of a molten flow portion of the polymer extruder, viii) a temperature for at least one bearing of a gear pump of the polymer extruder, ix) a temperature of an oil of the gear pump, x) an amperage of the gear pump, xi) a speed of the gear pump, xii) a suction pressure of the gear pump, xiii) a discharge pressure of the gear pump, xiv) a differential pressure of a screenpack of a die plate assembly of the polymer extruder, xv) a temperature of a die plate the die plate assembly, xvi) a temperature of polymer in the die plate, xvii) a pressure in the die plate, xviii) a speed of a pelletizer of the polymer extruder, xix) a ratio of power to amperage of the gear pump, or xx) combinations thereof.
